(19) United States
(12) Patent Application Publication
(10) Pub. No.: US 2022/0339103 A1
(43) Pub. Date: Oct. 27, 2022

(54) Lung-targeted corticosteroid treatment in viral respiratory disease, COVID-19 and ARDS (acute respiratory distress syndrome)

(71) Applicant: Bodor Laboratories, Inc., Miami, FL (US)

(72) Inventors: Nicholas Stephen BODOR, Miami, FL (US); Erik T BODOR, Miami, FL (US)

(21) Appl. No.: 17/724,474

(22) Filed: Apr. 19, 2022

Related U.S. Application Data
(60) Provisional application No. 63/248,162, filed on Sep. 24, 2021.
(60) Provisional application No. 63/176,418, filed on Apr. 19, 2021.

(51) Int. Cl. A61K 9/14; A61K 45/06; A61P 11/06; A61K 9/00; A61K 31/573; A61K 31/58

(52) U.S. Cl. CPC A61K 9/145; A61K 45/06; A61P 11/06; A61K 9/0078; A61K 31/573; A61K 31/58

(57) ABSTRACT

Provided herein are methods for treating, preventing, minimizing and/or substantially inhibiting inflammation associated with a respiratory virus such as COVID-19 and/or acute respiratory distress syndrome (ARDS), comprising administering to the lungs of a subject, by inhalation, a composition comprising an effective amount of an active agent such as a corticosteroid (for example, loteprednol etabonate (LE)). Also provided are pharmaceutical compositions comprising an active agent, such as a corticosteroid (for example, loteprednol etabonate (LE)) in an amount effective to treat, minimize and/or substantially inhibit inflammation associated with the respiratory virus, such as COVID-19 and/or ARDS.

### CROSS-REFERENCE TO RELATED APPLICATIONS

This application claims the benefit of U.S. Provisional Application No. 63/176,418, filed Apr. 19, 2021, and United States Provisional Application No. 63/248,162, filed Sep. 24, 2021, which are all incorporated by reference herein in their entireties and relied upon.

### FIELD

A method for treating, preventing, minimizing and/or substantially inhibiting inflammation associated with a respiratory virus such as COVID-19 and/or acute respiratory distress syndrome (ARDS), comprising administering to the lungs of a subject, by inhalation, a composition comprising an effective amount of an active agent such as a corticosteroid (for example, loteprednol etabonate (LE)). Also, pharmaceutical compositions comprising an active agent, such as a corticosteroid (for example, loteprednol etabonate (LE)) in an amount effective to treat, minimize and/or substantially inhibit inflammation associated with the respiratory virus, such as COVID-19 and/or ARDS.

### BACKGROUND

Patients with viral respiratory disease (e.g., COVID-19) often develop inflammatory responses that can lead to lung injury. It has been previously proposed that the potent anti-inflammatory effects of corticosteroids might prevent those effects. However, use of corticosteroids in patients with acute respiratory distress syndrome (ARDS) has resulted in both beneficial and deleterious clinical outcomes. High dose dexamethasone has been most frequently used and the 28-day mortality rate has been lower in patients randomized to corticosteroids, but serious side-effects have been observed. Moreover, serious, ophthalmic side-effects caused by dexamethasone have not been even followed.

### Corticosteroids in ARDS

COVID-19 has an initial period characterized by cough and fever, followed after around 8 days by development of dyspnea with pulmonary infiltration. Approximately a quarter of the patients admitted to hospital developed acute respiratory distress syndrome (ARDS) after a median of 10.5 days after symptom onset [1]. A JAMA editorial [2] concluded that sufficient data were accrued to issue a strong recommendation to treat patients with ARDS with corticosteroids. Oral or intravenous treatment was used, although some inhalation was also tried [3]. The DEXA-ARDS trial with moderate to severe ARDS patients found that high-dose dexamethasone reduced 60-day all-cause mortality (21% vs 36% P=0.005) [4]. A meta-analysis from the Rapid Appraisal for COVID-19 Therapies Working Group concluded that 28-day mortality was lower in patients randomized to corticosteroids vs. usual care or placebo. Administration of steroids is clearly associated with benefit among critically ill patients with COVID-19. The question still remains: what are the true incidence and optimal management of adverse effects? The possibility of harm could not be excluded. In addition to the well-known immunosuppressive effect, corticosteroids such as dexamethasone can cause, for example hyperglycemia, secondary infection, psychiatric effects, avascular necrosis, adrenal suppression, and the like; it is also to be noted that none of the COVID/ARDS studies looked into the well-known, serious ophthalmic side-effects: corticosteroids cause glaucoma [5] and cataract, particularly in steroid-sensitive patients [6]. The intraocular pressure (IOP) elevation caused by dexamethasone has been known for a long time [7]. It is important to note that corticosteroids are known to induce ocular hypertension when administered into the eye, or periocular, but also even systemic or inhalational routes. High doses of dexamethasone will certainly cause ocular hypertension and with time, cataract. In the elderly population, where some cataracts are already developed, relatively short-term treatment with high doses of dexamethasone will produce cataracts.

Corticosteroid treatment is helpful in the treatment of viral respiratory disease (e.g., COVID-19), mainly to alleviate inflammatory processes in the lung. During inhalation treatment [2], a significant portion of the inhaled dose can be swallowed and absorbed through soft tissues, and can reach the general circulatory system. Thus, particularly when high doses are used, administration of corticosteroids by inhalation can have similar effects as administration by other routes (e.g., systemic administration, oral administration, or administration by injection (e.g., intramuscular injection, intravenous injection, or subcutaneous injection)).

### SUMMARY

Considering the potential importance of using corticosteroids in ARDS, particularly in viral respiratory disease (e.g., COVID-19), provided herein is the use by inhalation of a therapeutic agent, such as a corticosteroid (for example, the retrometabolically designed, potent but very safe corticosteroid Loteprednol Etabonate (LE)), currently used widely in ophthalmology. Exemplary embodiments of this application include the following:

1. A method of treating, preventing, minimizing and/or substantially inhibiting inflammation associated with viral respiratory disease, (e.g., COVID-19) and/or acute respiratory distress syndrome (ARDS) in a mammalian subject in need of such treatment, said method comprising administering to the lungs of said subject, by inhalation, a composition comprising: (a) an effective amount of a therapeutic agent; and (b) a non-toxic pharmaceutically acceptable carrier therefor suitable for lung delivery by inhalation, wherein the therapeutic agent is selected from the group consisting of a compound of formula (I), a compound of formula (III) loteprednol etabonate (LE), and a combination thereof. In some embodiments, the therapeutic agent is loteprednol etabonate (LE).

2. The method of embodiment 1, wherein the therapeutic agent is micronized or submicronized.

3. The method of embodiment 1 or embodiment 2, wherein the average particle size of the therapeutic agent is about 0.01 to about 0.1 microns, about 0.1 microns to about 0.2 microns, about 0.2 microns to about 0.3 microns, about 0.3 microns to about 0.4 microns, about 0.4 microns to about 0.5 microns, about 0.5 microns to about 0.6 microns, about 0.6 microns to about 0.7 microns, about 0.7 microns to about 0.8 microns, about 0.8 microns to about 0.9 microns, about 0.9 microns to about 0.95 microns, about 0.95 microns to about 1.0 microns, about 0.01 microns, about 0.1 microns, about 0.2 microns, about 0.3 microns, about 0.4 microns, about 0.5 microns, about 0.6 microns, about 0.7 microns, about 0.8 microns, about 0.9 microns, or about 0.95 microns.

4. The method of any one of embodiments 1-3, wherein the ARDS is hyper-inflammatory ARDS.

5. The method of any one of embodiments 1-4, wherein the inflammation is inflammation associated with a cytokine storm.

6. The method of any one of embodiments 1-5, wherein the inflammation is interstitial inflammation.

7. The method of any one of embodiments 1-6, wherein the inflammation is alveolar inflammation.

8. The method of any one of embodiments 1-7, wherein the method comprises inhibiting at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10%, at least about 5%, of the inflammation.

9. The method of any one of embodiments 1-8, wherein the inflammation is treated, minimized and/or inhibited by an amount at least substantially equivalent to an amount that inflammation is treated, minimized and/or inhibited following administration by inhalation of a composition containing the same amount of a corticosteroid selected from among the group consisting of prednisone, prednisolone acetate, methylprednisolone, hydrocortisone, fludrocortisone, and dexamethasone.

10. The method of any one of embodiments 1-9, wherein administration of the composition results in reduced severity of a side effect compared to the severity of the side effect following administration by inhalation of a composition containing the same amount of a corticosteroid selected from among the group consisting of prednisone, prednisolone acetate, methylprednisolone, hydrocortisone, fludrocortisone, and dexamethasone.

11. The method of embodiment 10, wherein the side effect is selected from the group consisting of hyperglycemia, secondary infection, psychiatric effects, avascular necrosis, and adrenal suppression.

12. The method of one of embodiments 1-11, wherein said subject is infected with a COVID-19 virus.

13. The method of embodiment 12, wherein the COVID-19 virus is SARS-CoV-2.

14. The method of embodiment 12 or embodiment 13, wherein the subject is one who has been diagnosed with COVID-19.

15. The method of embodiment 14, wherein the composition is administered within about 24 hours, about 12 hours, about 6 hours, about 3 hours, about 1 hour, about 30 minutes, about 15 minutes, or about 5 minutes after the diagnosis of COVID-19.

16. The method of any one of embodiments 1-15, wherein said subject suffers from non-COVID-19 ARDS.

17. The method of embodiment 16, wherein the non-COVID-19 ARDS is associated with a condition selected from the group consisting of sepsis; inhalation of a harmful substance; severe pneumonia; viral infection, head, chest, or other major injury; pancreatitis; fat embolism; and lung transplantation.

18. The method of any one of embodiments 1-17, wherein the composition is administered prior to onset of severe lung symptoms.

19. The method of any one of embodiments 1-18, wherein the subject is one whose oxygenation of blood is at or above 90%.

20. The method of any one of embodiments 1-18, wherein the subject is one whose oxygenation of blood is below 90%.

21. The method of any one of embodiments 1-20, wherein the therapeutic agent is co-administered with an antibiotic.

22. The method of embodiment 21, wherein the antibiotic is selected from the group consisting of quinolone antibiotics, aminoglycosides, beta-lactams/beta-lactamase inhibitors, tetracyclines, fluoroquinolones, cephalosporins, carbapenems, and macrolide antibiotics.

23. The method of embodiment 21 or embodiment 22, wherein the antibiotic is selected from the group consisting of ciprofloxacin, ofloxacin, gemifloxacin, delafloxacin, tobramycin, carbenicillin, penicillin G, ticarcillin, ampicillin, nafcillin, cloxacillin, mezlocillin, oxacillin, piperacillin, azithromycin, amoxicillin, gentamicin and tobramycin, tetracycline, doxycycline, lymecycline, moxifloxacin, ceftriaxone, ornidazole, meropenem, piperacillin, tazobactam, piperacillin and tazobactam, vancomycin, ceftaroline, fluoroquinolone, and clarithromycin.

24. The method of any one of embodiments 21-23, wherein the antibiotic is administered by a route selected from the group consisting of systemic, oral and intravenous.

25. The method of any one of embodiments 21-24, wherein the antibiotic is administered before, after, or simultaneously with the composition comprising the therapeutic agent.

26. The method of any one of embodiments 1-25, wherein the therapeutic agent is co-administered with a corticosteroid.

27. The method of embodiment 26, wherein the corticosteroid is selected from the group consisting of prednisone, prednisolone acetate, methylprednisolone, hydrocortisone, fludrocortisone, dexamethasone, budesonide, ciclesonide, clobetasol propionate, fluticasone propionate, flunisolide, and betamethasone dipropionate.

28. The method of embodiment 26 or embodiment 27, wherein the corticosteroid is administered by a route selected from the group consisting of systemic, oral, and intravenous.

29. The method of any one of embodiments 26-28, wherein the corticosteroid is administered before, after, or simultaneously with the composition comprising the therapeutic agent.

30. The method of any one of embodiments 1-29, wherein the therapeutic agent is co-administered with an enhancer selected from the group consisting of cortienic acid, delta-1 cortienic acid, delta-1 cortienic acid methyl ester, and delta-1 cortienic acid ethyl ester.

31. The method of any one of embodiments 1-29, wherein the therapeutic agent is co-administered with an enhancer selected from the group consisting of hydrocortisone and hydrocortisone acetate.

32. The method of embodiment 30 or embodiment 31, wherein the enhancer is a micronized enhancer or a submicronized enhancer.

33. The method of any one of embodiments 30-32, wherein the enhancer is present in the composition comprising the therapeutic agent.

34. The method of any one of embodiments 30-32, wherein the enhancer is formulated in a separate composition.

35. The method of any one of embodiments 30-34, wherein the enhancer is administered before, after, or simultaneously with the composition comprising the therapeutic agent.

36. The method of any one of embodiments 1 to 35, wherein the therapeutic agent, and the co-administered enhancer, when it is present, are delivered to the lungs via a metered dose inhaler (MDI).

37. The method of any one of embodiments 1 to 35, wherein the therapeutic agent, and the co-administered enhancer when it is present, are delivered to the lungs via a dry powder inhaler (DPI).

38. The method of any one of embodiments 1 to 35, wherein the therapeutic agent, and the co-administered enhancer, when it is present, are delivered to the lungs via a nebulizer.

39. The method of embodiment 38, wherein the composition does not comprise a preservative.

40. A pharmaceutical composition, comprising: (a) a therapeutic agent in an amount effective to treat, prevent, minimize and/or substantially inhibit inflammation associated with viral respiratory disease, (e.g., COVID-19) and/or acute respiratory distress syndrome (ARDS) in a mammalian subject; and (b) a non-toxic pharmaceutically acceptable carrier therefor suitable for lung delivery by inhalation, wherein the pharmaceutical composition is formulated for administration to the lungs of the mammalian subject by inhalation, wherein the therapeutic agent is selected from the group consisting of a compound of formula (I), a compound of formula (III) loteprednol etabonate (LE), and a combination thereof. In some embodiments, the therapeutic agent is loteprednol etabonate (LE).

41. The pharmaceutical composition of embodiment 30, wherein the therapeutic agent is micronized or submicronized.

42. The pharmaceutical composition of embodiment 40 or embodiment 41, wherein the average particle size of the therapeutic agent is about 0.01 to about 0.1 microns, about 0.1 microns to about 0.2 microns, about 0.2 microns to about 0.3 microns, about 0.3 microns to about 0.4 microns, about 0.4 microns to about 0.5 microns, about 0.5 microns to about 0.6 microns, about 0.6 microns to about 0.7 microns, about 0.7 microns to about 0.8 microns, about 0.8 microns to about 0.9 microns, about 0.9 microns to about 0.95 microns, about 0.95 microns to about 1.0 microns, about 0.01 microns, about 0.1 microns, about 0.2 microns, about 0.3 microns, about 0.4 microns, about 0.5 microns, about 0.6 microns, about 0.7 microns, about 0.8 microns, about 0.9 microns, or about 0.95 microns.

43. The pharmaceutical composition of any one of embodiments 40-42, wherein the ARDS is hyper-inflammatory ARDS.

44. The pharmaceutical composition of any one of embodiments 40-42, wherein the inflammation is inflammation associated with a cytokine storm.

45. The pharmaceutical composition of any one of embodiments 40-44, wherein the inflammation is interstitial inflammation.

46. The pharmaceutical composition of any one of embodiments 40-45, wherein the inflammation is alveolar inflammation.

47. The pharmaceutical composition of any one of embodiments 40-46, wherein the therapeutic agent is present in an amount effective to inhibit at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10%, at least about 5%, of the inflammation.

48. The pharmaceutical composition of any one of embodiments 40-47, wherein the therapeutic agent is present in an amount effective to treat, minimize and/or inhibit inflammation by an amount at least substantially equivalent to an amount that inflammation is treated, minimized and/or inhibited following administration by inhalation of a composition containing the same amount of a corticosteroid selected from among the group consisting of prednisone, prednisolone acetate, methylprednisolone, hydrocortisone, fludrocortisone, and dexamethasone.

49. The pharmaceutical composition of any one of embodiments 40-48, wherein the therapeutic agent is present in an amount effective to treat, minimize and/or inhibit severity of a side effect compared to the severity of the side effect following administration by inhalation of a composition containing the same amount of a corticosteroid selected from among the group consisting of prednisone, prednisolone acetate, methylprednisolone, hydrocortisone, fludrocortisone, and dexamethasone.

50. The pharmaceutical composition of embodiment 49, wherein the side effect is selected from the group consisting of hyperglycemia, secondary infection, psychiatric effects, avascular necrosis, and adrenal suppression.

51. The pharmaceutical composition of any one of embodiments 40-50, further comprising an enhancer selected from the group consisting of cortienic acid, delta-1 cortienic acid, delta-1 cortienic acid methyl ester, and delta-1 cortienic acid ethyl ester.

52. The pharmaceutical composition of any one of embodiments 40-50, further comprising an enhancer selected from the group consisting of hydrocortisone and hydrocortisone acetate.

53. The pharmaceutical composition of embodiment 51 or embodiment 52, wherein the enhancer is a micronized enhancer or a submicronized enhancer.

54. The pharmaceutical composition of any one of embodiments 40-53, formulated as a metered dose inhaler (MDI).

55. The pharmaceutical composition of any one of embodiments 40-53, formulated as a dry powder inhaler (DPI).

56. The pharmaceutical composition of any one of embodiments 40-53, formulated as a nebulizer.

57. The pharmaceutical composition of embodiment 56, wherein the pharmaceutical composition does not comprise a preservative.

58. A combination, comprising the pharmaceutical composition of any one of embodiments 40-57, and an antibiotic.

59. A combination, comprising the pharmaceutical composition of any one of embodiments 40-57, and a corticosteroid.

60. A combination, comprising the pharmaceutical composition of any one of embodiments 40-57, an antibiotic, and a corticosteroid.

61. The combination of embodiment 58 or embodiment 60, wherein the antibiotic is selected from the group consisting of quinolone antibiotics, aminoglycosides, beta-lactams/beta-lactamase inhibitors, tetracyclines, fluoroquinolones, cephalosporins, carbapenems, and macrolide antibiotics.

62. The combination of any one of embodiments 58 and 60-61, wherein the antibiotic is selected from the group consisting of ciprofloxacin, ofloxacin, gemifloxacin, delafloxacin, tobramycin, carbenicillin, penicillin G, ticarcillin, ampicillin, nafcillin, cloxacillin, mezlocillin, oxacillin, piperacillin, azithromycin, amoxicillin, gentamicin and tobramycin, tetracycline, doxycycline, lymecycline, moxifloxacin, ceftriaxone, ornidazole, meropenem, piperacillin, tazobactam, piperacillin and tazobactam, vancomycin, ceftaroline, fluoroquinolone, and clarithromycin.

63. The combination of any one of embodiments 58 and 60-62, wherein the antibiotic is formulated for administration by a route selected from the group consisting of systemic, oral and intravenous.

64. The combination of any one of embodiments 58 and 60-63, wherein the antibiotic is formulated for administration before, after, or simultaneously with the composition comprising the therapeutic agent.

65. The combination of embodiment 59 or embodiment 60, wherein the corticosteroid is selected from the group consisting of prednisone, prednisolone acetate, methylprednisolone, hydrocortisone, fludrocortisone, dexamethasone, budesonide, ciclesonide, clobetasol propionate, fluticasone propionate, flunisolide, and betamethasone dipropionate.

66. The combination of any one of embodiments 59, 60 and 65, wherein the corticosteroid is formulated for administration by a route selected from the group consisting of systemic, oral, and intravenous.

67. The combination of any one of embodiments 59, 60, 65 and 66, wherein the corticosteroid is formulated for administration before, after, or simultaneously with the composition comprising the therapeutic agent.

68. A method of treating, preventing, minimizing and/or substantially inhibiting inflammation associated with COVID-19 and/or acute respiratory distress syndrome (ARDS) in a mammalian subject in need of such treatment, said method comprising administering to the lungs of said subject, by inhalation, a composition comprising: (a) an effective amount of a therapeutic agent; and (b) a non-toxic pharmaceutically acceptable carrier therefor suitable for lung delivery by inhalation, wherein the therapeutic agent is selected from the group consisting of a compound of formula (I), a compound of formula (III), loteprednol etabonate (LE), and a combination thereof

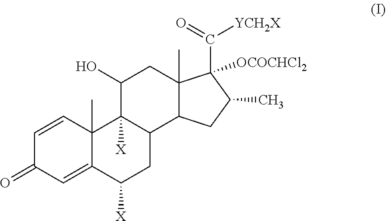

wherein, in Formula (I), each X is independently F or Cl, and Y is O or S; and

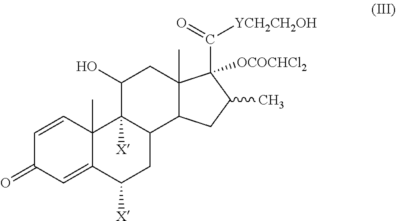

wherein, in Formula (III), X′ is independently H, F or Cl provided that at least one X′ is F or Cl, Y is O or S, and the wavy line indicates the α- or β-configuration.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to understand the disclosure and to see how it can be carried out in practice, embodiments will now be described, by way of non-limiting examples only, with reference to the accompanying drawings, in which:

FIG. 1 shows the formal design of Loteprednol Etabonate with designed-in actual metabolism, starting from the metabolite of prednisolone.

FIG. 2 shows major metabolic pathways of hydrocortisone.

FIG. 3 shows overlapping pharmacophore structures of loteprednol etabonate (black) and clobetasol propionate (gray).

FIG. 4 is a graph of competition curves for binding to rat lung cytosol glucocorticoid receptors (black diamond=LE; black square=dexamethasone; white diamond=Δ¹-cortienic acid etabonate (AE); “x”=Δ¹-cortienic acid (A).

FIG. 5 is a graph showing changes from baseline in the two-week average refletive Total Nasal Symptom Score (TNSS). Randomized, multi-center, parallel-group trial with 645 patients with seasonal allergic rhinitis (SAR) to grass pollen. Data are two-week average of the mean morning and evening TNSS based on reflective evaluation (±SEM). *LE treatment significantly more efficacious than PLA (p<0.03). (PLA=polylactic acid; LE100, LE200, LE400 and LE800=LE at a dose of 100 μg, 200 μg, 400 μg, and 800 μg, respectively).

### DETAILED DESCRIPTION

There is a need for treatments in which administration of corticosteroid produces a lung-specific local effect without inducing, or substantially inducing, systemic side-effects, such as, for example immunosuppression.

“Soft” steroids are compounds that can have potent anti-inflammatory activity (e.g., comparable with conventional steroids), but with minimal systemic activity. Examples of these compounds include Δ⁴ and Δ^{1,4} 17α-alkoxy-11β-hydroxy-3-oxoandrostenes optionally bearing various substituents at the 6, 9 and 16-positions and related 11-substituted compounds, which are esters or thioesters of 17β-carboxylic acids. 17α-ethers are described in U.S. Pat. No. 4,710,495. Some examples of compounds are haloalkyl esters of 17α-alkoxy-11β-hydroxyandrost-4-en-3-one-17β-carboxylic acids.

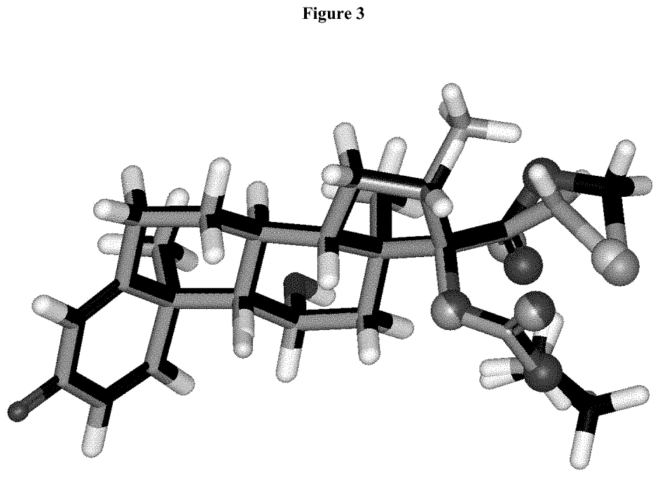

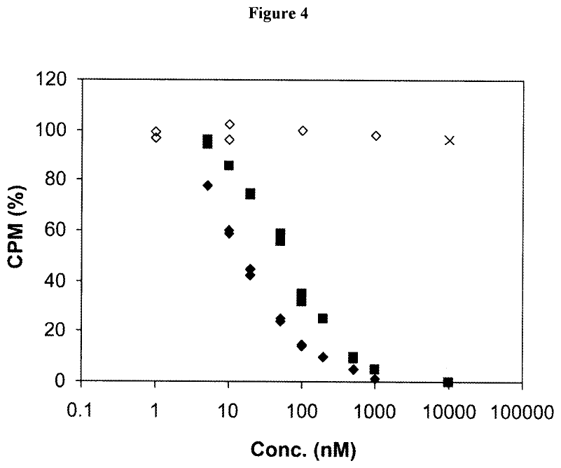

Some examples of “soft” steroids are 17α-carbonate-type soft corticosteroids. [9] Examples of 17α-carbonate-type soft corticosteroids are shown in the following Chemical Formula (V):

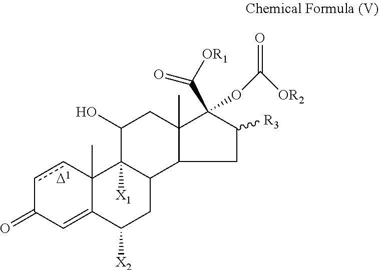

wherein R₁=halomethyl; R₂=alkyl, aryl, or aralkyl, R3=H, α- or (β-CH₃; X₁ and X₂ are independently selected from among H, F and Cl, and 66 ¹=double bond (present or absent). Examples of 17α-carbonate-type soft corticosteroids include haloalkyl 17α-alkoxycarbonyloxy-11β-hydroxyandrost-4-en-3-one-17β-carboxylates and the corresponding Δ1,4 compounds, optionally bearing 6α- and/or 9α-fluorine and 16α- or 16β-methyl substituents. One example of a 17α-carbonate-type soft corticosteroid is chloromethyl 17α-ethoxycarbonyloxy-11β-hydroxyandrosta-1,4-dien-3-one-17β-carboxylate, also known as loteprednol etabonate (LE). Loteprednol Etabonate (LE) can produce a lung-specific local effect without inducing, or substantially inducing, systemic side effects. LE is the active component of a number of ophthalmic products, and a uniquely safe and effective drug. Its corticosteroid receptor binding is significantly higher than that of dexamethasone. FIG. 3 shows the overlapping pharmacophore structures of LE and clobetasol propionate. FIG. 4 is a graph showing competition curves for binding to rat lung cytosol glucocorticoid receptors.

Other examples of corticosteroids are described in International Patent Publication No. WO2018232007A1, which is incorporated herein by reference in its entirety. Examples of corticosteroids include analogs of fluticasone.

Some examples of corticosteroids are a “soft” steroids having potent anti-inflammatory activity, comparable with conventional steroids, but with minimal systemic activity. Examples can include Δ⁴ and Δ^{1,4} 17α-alkoxy-11β-hydroxy-3-oxoandrostenes optionally bearing various substituents at the 6, 9 and 16-positions and related 11-substituted compounds, which are esters or thioesters of 17β-carboxylic acids. 17α-ethers are described in, for example, U.S. Patent No. 4,710,495. Some compounds are haloalkyl esters of 17α-alkoxy-11β-hydroxyandrost-4-en-3-one-17β-carboxylic acids.

Some ‘soft’ steroids, which can have potent anti-inflammatory activity and/or minimal systemic activity, are 17α-carbonates, such as described, for example, in U.S. Patent No. 4,996,335. These compounds include haloalkyl 17α-alkoxycarbonyloxy-11β-hydroxyandrost-4-en-3-one-17β-carboxylates and the corresponding Δ^{1,4} compounds, optionally bearing 6α- and/or 9α-fluorine and 16α- or 16β-methyl substituents. One of these compounds is chloromethyl 17α-ethoxycarbonyloxy-11β-hydroxyandrosta-1,4-dien-3-one-17β-carboxylate, also known as loteprednol etabonate (LE), approved by the FDA in 1998 and marketed worldwide in five or more products.

Etiprednol dicloacetate (ED; ethyl 17α-dichloroacetoxy-11β-hydroxyandrosta-1,4-diene-3-one-17β-carboxylate) is a second generation ‘soft’ corticosteroid, the first to contain halogen sub stituents in the 17α-position, which can serve as a pharmacophore. It has been shown that the dichloro function is necessary for activity (the monochloro derivative is void of activity, due to the unfavorable position the chlorine is forced into by steric hindrance). The dichloroacetyl functional group can contribute to the ‘soft’ nature of ED. Dichloro substituents can increase the second order rate constant kcat/kM of enzymatic hydrolysis in acetate esters by a factor of 20, compared to the unsubstituted ester while one chlorine substituent can cause no change or no substantial change.

Contrary to the first generation of ‘soft’ corticosteroids based on cortienic acid, represented by Loteprednol Etabonate (LE; 4) which can be hydrolytically deactivated by ester cleavage of the 17β-chloromethyl ester, in ED, the hydrolysis does not cleave at the 17β-ester, but primarily cleaves at the 17α-dichloroacetyl function. Nevertheless, the corresponding 17α-OH— metabolites can be inactive, thus contributing to soft drug properties.

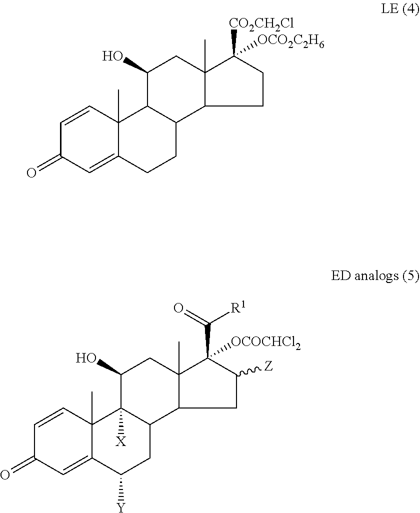

Some examples of corticosteroids include 17α-alkylcarbonyloxy-substituted corticosteroid compounds, such as, for example a compound of the formula (II):

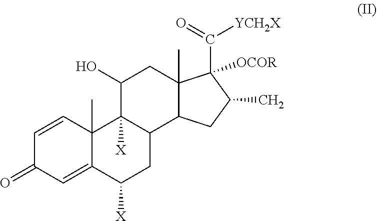

wherein each X is independently F or Cl, Y is O or S and R is C₁-C₃ alkyl. In some embodiments, Y is S. In some embodiments, said compound of formula (II) has local and/or systemic corticosteroid activity.

Some examples of corticosteroids are compound in which the 17α-OCOR group in formula (II) is replaced by a 17α-dichloroacetoxy (17α-OCOCHCl₂) group. In some embodiments, the corticosteroid is a soft corticosteroid compound of formula (I):

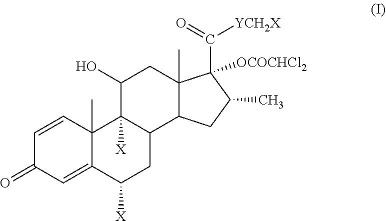

wherein each X is independently F or Cl, and Y is O or S. In some embodiments, Y is S. In some embodiments, said compound of formula (I) can have: 1) equivalent or substantially equivalent local corticosteroid activity as compared to the corresponding compound of formula (II); and/or 2) decreased, or substantially decreased, systemic corticosteroid activity as compared to the corresponding compound of formula (II).

In some embodiments, the compound of formula (I) can be selected from the group consisting of S-fluoromethyl 17α-dichloroacetoxy-6α,9α-difluoro-11β-hydroxy-16α-methyl-3-oxoandrosta-1,4-diene-17β-carbothioate, S-chloromethyl 17α-dichloroacetoxy-6α,9α-difluoro-11β-hydroxy-16α-methyl-3-oxoandrosta-1,4-diene-17β-carbothioate, fluoromethyl 17α-dichloroacetoxy-6α,9α-difluoro-11β-hydroxy-16α-methyl-3-oxoandrosta-1,4-diene-17β-carboxylate, and chloromethyl 17α-dichloroacetoxy-6α,9α-difluoro-11β-hydroxy-16α-methyl-3-oxoandrosta-1,4-diene-17β-carboxylate.

Among the compounds of formula (I), the closest analog of fluticasone propionate, i.e. the S-fluoromethyl compound just named, can have remarkable activity and/or desired hydrolytic susceptivity and thus can be a potent and/or safer alternative to fluticasone propionate. Fluticasone is currently one of several medications being investigated by Vanderbilt University and Duke University to evaluate its effectiveness in reducing symptoms of subjects with COVID-19.

Some examples of corticosteroids are a 17α-alkylcarbonyloxy-substituted corticosteroids, such as, for example, a compound of the formula (IV):

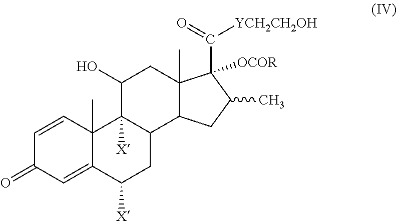

wherein each X is independently H, F or Cl provided that at least one X′ is F or Cl, Y is O or S, R is C₁-C₃ alkyl and the wavy line indicates the α- or β-configuration. In some embodiments, Y is S. In some embodiments, said compound of formula (IV) can have local and/or systemic corticosteroid activity.

Some examples of corticosteroids are compounds in which the 17α-OCOR group in formula (IV) is replaced with a 17α-dichloroacetoxy (17α-OCOCHCl₂) group. In some embodiments, the corticosteroid is a soft corticosteroid compound of formula (III):

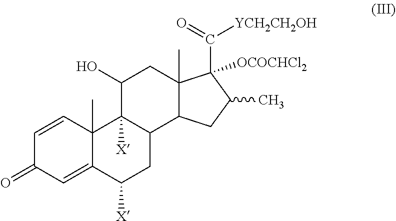

wherein each X′ and Y are as defined with formula (IV) above, and the wavy line indicates the α- or β-configuration. In some embodiments, Y is S. In some embodiments, said compound of formula (III) can have: 1) equivalent or substantially equivalent local corticosteroid activity as compared to the corresponding compound of formula (IV); and/or 2) decreased or substantially decreased, systemic corticosteroid activity as compared to the corresponding compound of formula (IV).

In some embodiments, the compound of formula (III) is selected from the group consisting of 2-hydroxyethyl 17α-dichloroacetoxy-6α,9α-difluoro-11β-hydroxy-16β-methyl-3-oxoandrosta-1,4-diene-17β-carboxylate and 2-hydroxyethyl 17α-dichloroacetoxy-9α-fluoro-11β-hydroxy-16β-methyl-3-oxoandrosta-1,4-diene-17β-carboxylate.

In some embodiments, the compound (I) or (III) can comprise a potency-increasing substitution at the 6α, 9α and 16α-positions of the compound (II) or (IV) while replacing the 17α-OCOR group with a 17α-dichloroacetoxy (17α-OCOCHCl₂) group.

Some examples of corticosteroids include the following compound 25, which is a soft analog of fluticasone propionate, where the 17α-propionyl function is replaced by the hydrolytically more labile dichloroacetyl function:

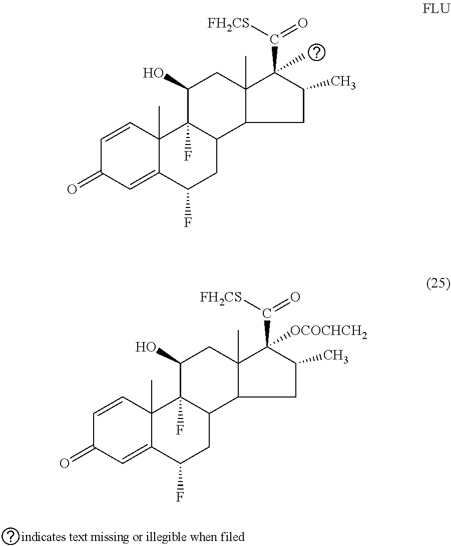

Some examples of corticosteroids include compounds of the following Formula (VI), in which X, Y, Z, and R′ are selected according to the table below:

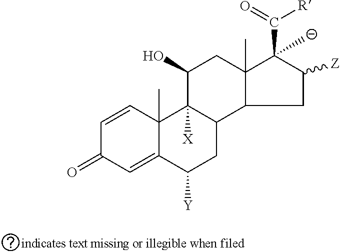

| Compound | X | Y | Z | R′ |
|---|---|---|---|---|
| Etiprednol | H | H | H | OCH₂CH₃ |
| Dicloacetate | | | | |
| VI-a | F | H | β-CH₃ | OCH₃ |
| VI-b | F | H | β-CH₃ | OCH₂CH₃ |
| VI-c | F | H | β-CH₃ | OCH₂Cl |
| VI-d | F | H | β-CH₃ | OCH₂CH₂OH |
| VI-e | F | F | β-CH₃ | OCH₃ |
| VI-f | F | F | β-CH₃ | OCH₂CH₃ |
| VI-g | F | F | β-CH₃ | OCH₂Cl |
| VI-h | F | F | β-CH₃ | OCH₂CH₂OH |
| VI-i | F | F | α-CH₃ | OCH₂F |
| VI-j | F | F | α-CH₃ | OCH₂Cl |
| VI-k | F | F | α-CH₃ | SCH₂F |
| VI-l | F | F | α-CH₃ | SCH₂Cl |

Provided herein are compositions, uses, and methods for treating, preventing, minimizing and/or substantially inhibiting inflammation associated with acute respiratory distress syndrome. The compositions, uses, and methods provided herein can employ a corticosteroid described herein, such as, for example a compound of formula (I), a compound of formula (III), LE, or a combination thereof. The compositions, uses, and methods provided herein can employ a corticosteroid described herein, such as, for example a compound of formula (I), a compound of formula (III), LE, or a combination thereof, and another corticosteroid. In some embodiments, the compositions, uses, and methods provided herein can employ LE. In some embodiments, the compositions, uses, and methods provided herein can employ LE in combination with another corticosteroid. In some embodiments, treating ARDS comprises treating the inflammatory component of ARDS. In some embodiments, the inflammatory response is an inflammatory response in the lungs and/or bronchi. In some embodiments, the corticosteroid (e.g., LE) is formulated for administration by inhalation. It is understood that the compositions, uses, and methods described herein are not limited to employing LE, and can employ, LE and/or another corticosteroid described herein. In some embodiments, the corticosteroid is a compound of formula (I) or formula (III). In some embodiments, the corticosteroid is a compound of formula (I) or formula (III) in which Y═S. In some embodiments, the corticosteroid is S-fluoromethyl 17α-dichloroacetoxy-6α,9α-difluoro-11β-hydroxy-16α-methyl-3-oxoandrosta-1,4-diene-17β-carbothioate. In some embodiments, the corticosteroid is 2-hydroxyethyl 17α-dichloroacetoxy-6α,9α-difluoro-11β-hydroxy-16β-methyl-3-oxoandrosta-1,4-diene-17β-carboxylate.

Combination therapy of a corticosteroid and another therapy may be more effective than monotherapy for treating COVID-19. However, administering corticosteroids prior to other therapies (e.g., antiviral drugs) and/or prior to (or in the early stages of) symptom onset, could aggravate respiratory disease severity (for example, in COVID-19 patients). Thus, in some examples, it may be advisable to administer a corticosteroid after other therapies (e.g., antiviral drugs) and/or after the early phase of the disease. Shionoya et al. (2021) PLoS ONE 16 (9): e0256977, https://doi.org/10.1371/journal.pone.0256977. For example, administration of antiviral drugs in the viral response phase and corticosteroids in the host inflammatory response phase may be a preferred method of administration. Administration of a corticosteroid could delay recovery and/or increase severity if administered too early, for example as a preventive measure, especially the viraemia phase. Arora et al. (2021) BMJ Case Rep. 14:e241105.

### Loteprednol Etabonate, a Retrometabolically Designed Safe Corticosteroid

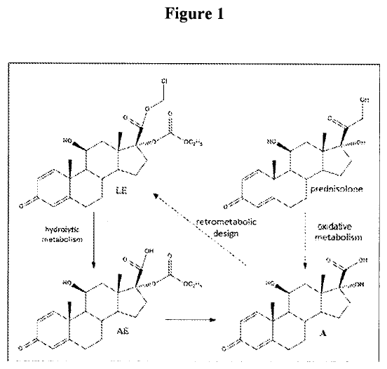

Retrometabolic drug design [8] strategically combines structure-activity (SAR) and structure-metabolism (SMR) relationships, allowing effective separation of drug action and side-effects, resulting in a significant improvement of the therapeutic index TI (TD50/ED50—ratio of toxic and effective doses). One design approach is not only to study the metabolism but to build into the drug molecule the desired metabolism, in addition to the therapeutic activity. The objective is not to avoid metabolism, but direct its chemistry and timing. One basic principle is to avoid oxidative metabolic transformations (relatively slow, subject to competition and generate toxic intermediates, radicals) and replace them with hydrolytic processes. This can be achieved for example, by starting the design process with an inactive metabolite of a drug, the product of an oxidative process, which is then converted by isosteric/isoelectronic modifications into an active ‘soft’ drug, of which strategically modified molecular structure dictates its hydrolytic metabolism back to the inactive metabolite the drug design started with. As applied to LE (shown in FIG. 1), its design starts with delta-1-cortienic acid A, an inactive metabolite of prednisolone. The original 17-alpha-dihydroxyacetone pharmacophore of prednisolone is replaced by the chloromethyl ester of the delta-1-cortienic acid. The customary 17-alpha-ester (an activity enhancer) is replaced with a novel type of substituent, the 17-alpha-ethyl carbonate, which prevents formation of a toxic, reactive intramolecular mixed anhydride.

The resulting LE [9] was found to be significantly more potent than prednisolone acetate and even dexamethasone, but is void of any systemic activity or side-effects. FIG. 4 shows a graph of competition curves for binding to rat lung cytosol glucocorticoid receptors by LE, dexamethasone, Δ¹-cortienic acid etabonate (AE), and Δ¹-cortienic acid (A) [8]. LE is also void of the debilitating ophthalmic side-effects, elevation of IOP (intra-ocular pressure) (glaucoma) [10] and cataract (due to the lack of the 20-keto function, responsible for the cataract forming protein-steroid adducts). In one study [25], subjects (healthy volunteers or patients with inflammation or allergy) in all sponsored loteprednol etabonate studies in the United States were evaluated. A clinically significant elevation in IOP was defined as greater than or equal to 10 mmHg at any visit, and long-term use was defined as greater than or equal to 28 days. Of the 2,210 subjects, 1,648 were treated for 28 days or longer with LE (0.2% or 0.5%), prednisolone acetate 1%, or vehicle. IOP elevation greater than or equal to 10 mmHg occurred in 0.64% (4/624) of subjects taking long-term loteprednol etabonate, 0.99% (3/304) of subjects taking vehicle, and 6.71% (11/164) of subjects taking prednisolone acetate, respectively. In a retrospective review [26] of 397 patients being treated long-term with 0.2% loteprednol etabonate ophthalmic suspension (>1 yr: n=159; >2 yr: n=84; and >3 yr: n=22), there were no reported adverse effects of long-term loteprednol etabonate use in any of the 159 patients, whose continuous use ranged from a cumulative total of 120 drops per eye to 3,741 drops per eye.

LE was approved by the FDA for four different ophthalmic inflammatory and allergic diseases and has been marketed since 1998. A recent extensive review [11], discussing 32 different clinical trials demonstrates the unique selective activity and lack of side-effects of LE. LE has a high level of corticosteroid-related efficacy and potency, and no or little IOP elevation in animal models [11]. Known formulations of LE include ophthalmic suspensions, an ointment, a combination product with tobramycin, and a gel [11]. LE demonstrates efficacy in ocular inflammatory diseases (e.g., giant papillary conjunctivitis, seasonal allergic conjunctivitis, vernal keratoconjunctivitis, anterior uveitis, blepharokeratoconjunctivitis, dry eye disorders, and combinations thereof) and for control of postoperative inflammation and pain following cataract surgery and refractive surgeries [11].

The unique value of LE is also presented by replacing prednisolone acetate (PA) with LE in a group of corneal transplant patients developing very high IOP. By replacing PA with LE, all IOP-s were returned to normal, without compromising the desired therapeutic effect [12]. Clinically significant IOP elevation following administration of LE is low (similar to vehicle), and significantly less than with other corticosteroids, even in patients who are steroid responders [11]. As a soft corticosteroid, LE would, perform similarly in other local applications. Its remarkable therapeutic index was first established by the classical subcutaneous cotton pellet granuloma test (see ref. [8]), as shown in Table 1. Human vasoconstrictor studies [13] demonstrated very good topical activity.

TABLE 1
Therapeutic index comparison of corticosteroids^{a}

| Steroid | n | ED₅₀^{b} | Rel.pot.^{c} | TD₅₀^{d} | Rel.pot.^{e} | TI^{f} |
|---|---|---|---|---|---|---|
| Loteprednol | 8 | 178.0 | 0.48 | 10,000 | 0.02 | 24.0 |
| Etabonate (0.1%) | | | | | | |
| Hydrocortisone 17α- | 8 | 121.0 | 0.70 | 369 | 0.57 | 1.3 |
| butyrate (HB) (0.1%) | | | | | | |
| Betamethasone 17α- | 8 | 84.8 | 1.00 | 212 | 1.00 | 1.0 |
| valerate (BV) (0.12%) | | | | | | |
| Clobetasone-17α- | 8 | 2.9 | 29.70 | 11 | 19.30 | 1.5 |
| propi onate (KP) | | | | | | |
| (0.1%) | | | | | | |

^{a}Subcutaneous cotton pellet granuloma test.
^{b}Anti-inflammatory activity in the cotton pellet granuloma test (μg/pellet)
^{c}Relative inflammatory effect compared to betamethasone.
^{d}Thymolysis potency (μg/pellet).
^{e}Relative timolysis effect.
^{f}Therapeutic index: rel.pot.^{c}/rel.pot.^{a}

More relevant for the present purpose, LE was found to be very effective and safe in preliminary human tests [14] and in phase II/III studies of seasonal allergic rhinitis [15].

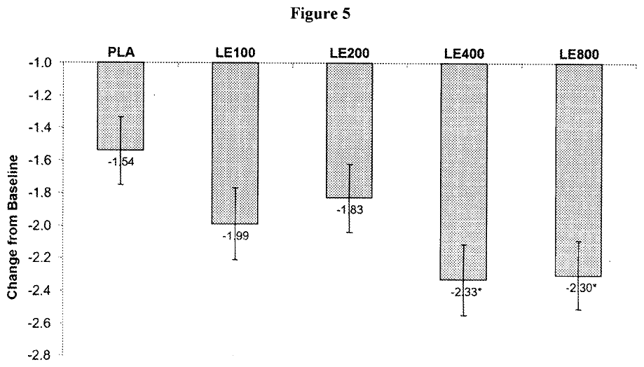

A dose-ranging SAR study showed that administration of an LE nasal spray was more efficacious than administration of polylactic acid (FIG. 5).

There was no significant suppression of the hypothalamic-pituitary axis at any Loteprednol Etabonate use. The robust improvement in the therapeutic index when LE was administered intrapulmonally [16], is shown in Table 2. Table 2 shows the ratios of intrapulmonal doses which induced moderate, about 60% inhibition of late phase eosinophilia in actively sensitized and challenged Brown Norway rats and those which caused significant reduction of the thymus following intrapulmonal drug administration on five consecutive days. The potential use by inhalation in asthma was well justified [17].

TABLE 2
Ratios between the lowest “side-effect-inducing” dose¹ and a moderately effective therapeutic dose²

| Compounds | Ratios |
|---|---|
| Beclomethasone | 0.3 |
| Dipropionate | |
| Budesonide | 2 |
| Fluticasone | 2 |
| Loteprednol Etabonate | 40 |

¹The dose which causes a significant reduction of thymus weight in Brown Norway rats.
²The dose which causes about 50-60% inhibition of airway eosinophilia in actively sensitized Brown Norway rats.

It was just recently discovered [18], that LE is hydrolyzed by the HDL-connected Paraoxonase 1. Because this esterase is not found in the lung, LE is not hydrolyzed in the lung, rather it is not hydrolyzed until it reaches the bloodstream. As a result, the LE is particularly long-acting when administered by inhalation.

Loteprednol Etabonate (LE) is currently manufactured by a number of companies and is available as a micronized (average 5 micron) crystalline powder. For ophthalmic applications aqueous suspensions of 0.25% and 0.5% concentrations are used. Similar formulations were very effective for the treatment of rhinitis. There are two companies in the US selling LE suspension, gel and cream formulations (see, Table 3 below).

TABLE 3
Formulations of Loteprednol Etabonate

| Brand Name | Formulation | Date of FDA Approval |
|---|---|---|
| Lotemax ® | loteprednol etabonate 0.5% | Mar. 9, 1998 |
| | ophthalmic suspension | |
| Alrex ® | loteprednol etabonate 0.2% | Mar. 9, 1998 |
| | ophthalmic suspension | |
| Zylet ® | loteprednol etabonate 0.5% + | Dec. 14, 2004 |
| | tobramycin 0.3% ophthalmic | |
| | suspension | |
| Lotemax | loteprednol etabonate 0.5% | Apr. 15, 2011 |
| Ointment ® | ointment | |
| Lotemax Gel ® | loteprednol etabonate 0.5% | Sep. 28, 2012 |
| | gel | |
| Inveltys | loteprednol etabonate 1.0% in | Aug. 22, 2018 |
| | mucus penetrating | |
| | nanoparticles (MPP) | |
| KP - 121 | loteprednol etabonate 0.25% | |
| | MPP for dry eye | |

The aqueous suspensions can be adapted for nebulizing, although capsules formulated for nebulizers can also be developed. Powder inhalation is also feasible, a number of such steroid formulations are currently used, where the active ‘hard’ conventional corticosteroid could be replaced by LE, to avoid the side-effects. A powder formulation for a new soft steroid, Etiprednol Dicloacetate [19] was developed; a corticosteroid (e.g., LE) can be formulated similarly.

Methods by which corticosteroids can be delivered to the lung include, for example metered dose inhalers (MDIs), dry powder inhalers (DPIs), and nebulizers. MDIs can have the drug either dissolved or suspended in a liquified, pressurized propellant. Pressing the actuator button briefly releases the pressure, converting a measured amount of propellant into aerosol delivered from the orifice. Older MDIs used chlorofluorocarbons (CFCs) as the propellant, these have been phased out in favor of hydrofluoroalkanes (HFAs) today. MDIs require the patient to inspire as the button is pressed.

With DPIs, the patient's own inspiration provides the energy for drug delivery. The breath draws air through the drug, formulated as a dry powder.

In the above cases, the therapeutic agent (e.g., LE) can be micronized (1-5 microns) and mixed with a carrier, most frequently alpha-lactose (a-L), of larger particles. (Lactose is 4-(b-D-galactosido-)-D-glucose). This can allow easily controllable doses. A dose can generally contain 12.5-25 mg a-L, intimately mixed with the steroid dose of 50-800 micrograms (0.1-0.8 mg). The drug is loosely attached to the a-L surface and the airflow separates it. Particles of 1-5 microns can reach the lungs, while the large carrier particles cannot.

The third delivery way is use of nebulizers. These convert a liquid suspension or solution into an aerosol using a jet of compressed air or ultrasonic energy. The aerosol plume is then delivered to the patient through either a face mask or a mouthpiece. These devices make minimal demands on the patient technique and are normally used when the patient is unable to use MDIs or DPIs. Nebulizers are often used in a hospital setting. Standard nebulizers use ampules of 2 ml of the drug (0.25-2 mg LE) in a suspension containing some disodium edetate, sodium chloride, sodium citrate, citric acid, and polysorbate 80 in water.

In addition, the ophthalmic suspension of LE sold as Lotemax (TM) could be nebulized and used in the capsules. The current LE API (active pharmaceutical ingredient) available is micronized to 2-5 microns and used as suspension.

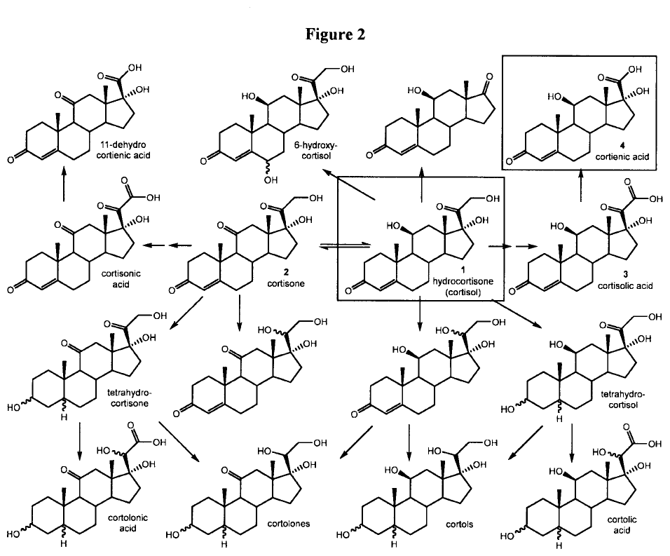

Still further, the therapeutic agent (e.g., a compound of formula (I), a compound of formula (III), loteprednol etabonate (LE), or a combination thereof) can be co-administered with an enhancing agent (also referred to as an “enhancer”), such as, for example, as described in Bodor U.S. Pat. No. 7,560,448 B2 and Bodor U.S. Pat. No. 7,687,484B2, both of which are incorporated by reference herein in their entireties and relied upon. Among the enhancers provided by the 7,560,448 patent, preferred ones for use herein with LE include cortienic acid, delta-1 cortienic acid, delta-1 cortienic acid methyl ester and delta-1 cortienic acid ethyl ester. Among the enhancers provided by the U.S. Pat. No. 7,687,484 patent, preferred ones for use herein with LE include hydrocortisone and hydrocortisone acetate. Examples of major metabolic pathways of hydrocortisone are shown in FIG. 2. [17] The selected enhancer can be used in micronized or submicronized form, like LE, so that it can be delivered to the lungs. There are examples of specific formulations in these patents. The enhancer is typically added to same composition as the LE and the carrier for co-administration herein.

Throughout this specification, the following definitions, general statements and illustrations are applicable.

The patents, published applications and scientific literature referred to herein establish the knowledge of those with skill in the art and are hereby incorporated by reference in their entireties to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used herein, whether in a transitional phrase or in the body of a claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a composition, the term “comprising” means that the composition includes at least the recited features or components, but may also include additional features or components.

The terms “consists essentially of” or “consisting essentially of” have a partially closed meaning, that is, they do not permit inclusion of steps or features or components which would substantially change the essential characteristics of a process or composition; for example, steps or features or components which would significantly interfere with the desired properties of the compounds or compositions described herein, i.e., the process or composition is limited to the specified steps or materials and those which do not materially affect the basic and novel characteristics of the process or composition.

The terms “consists of” and “consists” are closed terminology and allow only for the inclusion of the recited steps or features or components.

As used herein, the singular forms “a,” “an” and “the” specifically also encompass the plural forms of the terms to which they refer, unless the content clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

As used herein, the recitation of a numerical range for a variable is intended to convey that the variable can be equal to any values within that range. Thus, for a variable which is inherently discrete, the variable can be equal to any integer value of the numerical range, including the end points of the range. Similarly, for a variable which is inherently continuous, the variable can be equal to any real value of the numerical range, including the end points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

In the specification and claims, the singular forms include plural referents unless the context clearly dictates otherwise. As used herein, unless specifically indicated otherwise, the word “or” is used in the “inclusive” sense of “and/or” and not the “exclusive” sense of “either/or.”

Technical and scientific terms used herein have the meaning commonly understood by one of skill in the art to which the present description pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10th Ed., McGraw Hill Companies Inc., New York (2001), as well as more recent editions.

As used herein, “treating” means reducing, hindering or inhibiting the development of, controlling, inhibiting, alleviating and/or reversing the symptoms in the subject to which a composition comprising a corticosteroid (e.g., a compound of formula (I), a compound of formula (III), LE, or a combination thereof) has been administered, as compared to the symptoms of a subject not being administered the compound or composition. A practitioner will appreciate that the combinations, compositions, dosage forms and methods described herein are to be used in concomitance with continuous clinical evaluations by a skilled practitioner (physician or veterinarian) to determine subsequent therapy. Such evaluation will aid and inform in evaluating whether to increase, reduce or continue a particular treatment dose, and/or to alter the mode of administration.

The subject compounds or compositions can also prevent the symptoms, or prevent the occurrence of the symptoms, in the subject to which a composition comprising a therapeutic agent (e.g., a compound of formula (I), a compound of formula (III), LE, or a combination thereof) has been administered, as compared to the symptoms of a subject not being administered the compound or composition. This is not a prevention of viral respiratory disease (e.g., COVID-19) infection or other ARDS in the absolute sense; it does not prevent the medical condition, as it does not even address the condition's cause; rather it inhibits the manifestation of the condition for the period of time (e.g., hours) for which the administered dose is effective.

The methods described herein are intended for use with any mammalian subject/patient that may experience their benefits. Thus, the terms “subjects” as well as “patients,” “individuals”, “warm-blooded animals” and “mammals” include humans as well as non-human subjects, such as animals that may experience viral respiratory disease (e.g., COVID-19 infection) or other ARDS.

In conclusion, inhaled corticosteroid (e.g., a compound of formula (I), a compound of formula (III), Loteprednol Etabonate, or a combination thereof) can be provided to be used to treat ARDS/viral respiratory disease (e.g., COVID-19).

Two important reasons why this treatment will remain highly relevant even after a successful general distribution of COVID-19 vaccine, in addition to a specific new aspect, are as follows:

First, while the unusually fast development of several vaccines against COVID-19 is excellent news, it unfortunately does not eliminate COVID-19 itself. It is expected that this virus (and related strains) will be with us for years to come, making the provided treatment relevant for future cases. A second, very important consideration is that ARDS is not specific to COVID-19. There are many underlying causes of ARDS, such as sepsis, inhalation of harmful substances (chemical fumes, vomit, and others), severe pneumonia, infection (e.g., viral infection such as injection by H1N1 influenza or SARS-CoV-2), head, chest or other major injuries, pancreatitis, embolism (e.g., fat embolism), lung transplantation, to name a few. To demonstrate this, the March 2020 study previously referenced [4] included 277 pre-pandemic ARDS patients, where use of dexamethasone decreased all cause mortality by 15% and resulted in significantly more ventilator-free days. This illustrates a clear ongoing need for treatment of ARDS. In addition to dexamethasone, other corticosteroids are useful. For example, prolonged use of low-dose methylprednisolone was effective [20] in non-COVID-19 ARDS patients. Also, hydrocortisone and fludrocortisone were effective in septic shock [21] or in pneumonia [22].

Thus, corticosteroids can be used to treat, prevent, substantially prevent, dampen and/or suppress a cytokine storm, thereby reducing lung damage (the cytokine storm can also be triggered by H1N1 influenza). However, due to the serious side-effects of dexamethasone, it has been proposed [23] to use it in intermittent ‘pulse’ doses, followed by nebulized triamcinolone, in order to concentrate the effect within the lungs only. This further emphasizes the importance of the use of inhaled corticosteroid (e.g., a compound of formula (I), a compound of formula (III), Loteprednol Etabonate, or a combination thereof), such as Loteprednol Etabonate (the safest potent corticosteroid), instead, as among other advantages, its proven high therapeutic index would make such precautionary dosage methods unnecessary.

Based on all known evidence, in order to prevent and treat cytokine storms of viral respiratory disease (e.g., COVID-19) or any other origin, it is recommended to start use of nebulized or powder-inhaled corticosteroid (e.g., a compound of formula (I), a compound of formula (III), Loteprednol Etabonate, or a combination thereof) as prophylaxis/treatment as soon as possible, immediately after diagnosis. Treatment at home before hospitalization becomes necessary can obviate the need for hospitalization entirely and prevent the serious progression in decreasing lung function that characterizes COVID-19 infection and ARDS in other conditions.

Treatment with a corticosteroid (e.g., a compound of formula (I), a compound of formula (III), Loteprednol Etabonate, or a combination thereof) is safe and effective for long-term use in this way. It can thus be concentrated onto the lungs—the most important organ affected by the cytokine storm.

### REFERENCES

1. Huang, C., Y. Wang et al., Clinical Features of Patients Infected with 2019 Novel Coronavirus in Wuhan China. Lancet, 2020, 395: p. 497-506.
2. Prescott, H. C., T. W. Rice, Corticosteroid in COVID-19 ARDS. Evidence and Hope During the Pandemic. JAMA 2020, 324 (13): p. 1292-1295.
3. Halpin M. G., D. Singh and R. M. Hadfield, Inhaled Corticosteroids and COVID-19: A Systematic Review and Clinical Perspective. Eur Resp J, 2020, 55, 2001009.
4. Villar, J., C. Ferrando et al. Dexamethasone in ARDS Network. Dexamethasone Treatment for the Acute Respiratory Distress Syndrome: A Multicentre, Randomized, Controlled Trial, Lancet Respir Med, 2020, 8 (3): p. 267-276.
5. Phulka, S., S. Kaushik et al., Steroid-induced Glaucoma: An Avoidable Irreversible Blindness. J Curr Glaucoma Pract, 2017, 11 (2): p. 67-72.
6. Becker, B., Intraocular Pressure Response to Topical Corticosteroids. Invest Ophthalmol Vis Sci, 1965, 4: p. 198-205.
7. Armaly, M. F., The Heritable Nature of Dexamethasone-Induced Ocular Hypertension. Arch Ophthalmol, 1966, 75 (1): p 32-35.
8. Bodor, N., P. Buchwald, Retrometabolism-Based Drug Design and Targeting. In Burger's Medicinal Chemistry, Vol 2: Drug Discovery and Development, 6th Edition, John Wiley and Sons: New York, Ed. D. Abraham, 2003, 2 (15): p. 533-608.
9. Bodor, N., Soft Steroids Having Antiinflammatory Activity, U.S. Pat. No. 4,996,335, Feb. 26, 1991.
10. Bodor, N., Nicole Bodor, W. Wu, A Comparison of Intraocular Pressure Elevating Activity of Loteprednol Etabonate and Dexamethasone in Rabbits. Cur Eye Res, 1992, 11 (6): p 525-530.
11. Comstock, T. L., J. D. Sheppard, Loteprednol Etabonate for Inflammatory Conditions of the Eye: Twenty Years of Clinical Experience with a Retrometabolically Designed Corticosteroid. Exp Opin Pharmacother, 2018, 13: p. 1427-1438.
12. Holland, E. J. et al., Attenuation of Ocular Hypertension with the Use of Topical Loteprednol Etabonate 0.5% in Steroid Responders After Corneal Transplantation. Cornea, 2009, 28: p. 1139-1143.
13. Bodor, E. T., W.-M Wu et al., Enhanced Activity of Topical Hydrocortisone by Competitive Binding of Corticosteroid-Binding Globulin. J Pharm Sci, 2016, 105: p. 2873-2878.
14. Geldmacher, H., A. Buchendahl et al., A Pilot Study to Assess the Efficacy of Loteprednol Etabonate Nasal Spray as a Treatment for Allergic Rhinitis in an Experimental Exposure Unit (EEU). Allergy, 2002, 57 (Suppl. 73): p. 234.
15. Krug, N., J. M. Hohfield et al., Effects of Loteprednol Etabonate Nasal Spray Suspension on Seasonal Allergic Rhinitis Assessed by Allergen Challenge in an Environmental Exposure Unit. Allergy, 2005, 60 (3): p. 354-359.
16. Szelenyi, I., R. Hermann et al., Possibilities in Improvement of Glucocorticoid Treatment in Asthma with Special Reference to Loteprednol Etabonate. Pharmazie, 2004, 59: p. 409-411.
17. Bodor, N., P. Buchwald, Corticosteroid Design for the Treatment of Asthma: Structural Insights and the Therapeutic Potential of Soft Corticosteroids. Curr Pharm Design, 2006, 12: p. 3241-3260.
18. Samir, A., N. Bodor, T. Imai, Identification of the Esterase Involved in the Metabolism of Two Corticosteroid Soft Drugs. Biochem Pharmacol, 2017,127: p. 82-89.
19. Kurucz, I., S. Meszaros et al., Anti-inflammatory Effect and Soft Properties of Etiprednol Dicloacetate, a New Antiasthmatic Soft Steroid. Pharmazie, 2004, 5: p. 412-416.
20. Meduri, G., R. Siemieniuk, R. Ness, S. Seyler, Prolonged Low-Dose Methylprednisolone Treatment is Highly Effective in Reducing Duration of Mechanical Ventilation and Mortality in ARDS. J Intensive Care, 2018, 6:53, doi:10.1186/s40560-018-0321-9.
21. Venkatesh, B., S. Finfer, J. Cohen et al., Hydrocortisone Plus Fludrocortisone for Adults with Septic Shock. N Engl J Med, 2018, 378 (9): p. 809-818.
22. Stern, A., K. Skalsky, T. Arni et al., Corticosteroids for Pneumonia. Cochrane Database Syst Rev, 2017, 12: ED007720.
23. Sharum, K., R. Tiwari et al., Dexamethasone to Combat Cytokine Storm in COVID-19: Clinical Trials and Preliminary Evidence. Int J Surgery, 2020, 82: p. 179-181.
24. Bodor, N., Buchwald, P. Curr. Med. Chem. 2001, 8 (2), cover.
25. Novack, G., Howes, J., Crockett, R., Sherwood, M., Change in intraocular pressure during long-term use of loteprednol etabonate J. Glaucoma, 1998 7 (4):266-269.
26. Ilyas, H., Slonim, C., Braswell, G., Favetta, J., Schulman, M., Long-term Safety of Loteprednol Etabonate 0.2% in the Treatment of Seasonal and Perennial Allergic Conjunctivitis, Eye Contact Lens, 2004 30 (1):10-13.

What is claimed:

1. A method of treating, preventing, minimizing and/or substantially inhibiting inflammation associated with COVID-19 and/or acute respiratory distress syndrome (ARDS) in a mammalian subject in need of such treatment, said method comprising administering to the lungs of said subject, by inhalation, a composition comprising: (a) an effective amount of loteprednol etabonate (LE) and (b) a non-toxic pharmaceutically acceptable carrier therefor suitable for lung delivery by inhalation.

2. The method of claim 1, wherein the LE is micronized LE or submicronized LE.

3. The method of claim 1, wherein the average particle size of the LE is about 0.01 to about 0.1 microns, about 0.1 microns to about 0.2 microns, about 0.2 microns to about 0.3 microns, about 0.3 microns to about 0.4 microns, about 0.4 microns to about 0.5 microns, about 0.5 microns to about 0.6 microns, about 0.6 microns to about 0.7 microns, about 0.7 microns to about 0.8 microns, about 0.8 microns to about 0.9 microns, about 0.9 microns to about 0.95 microns, about 0.95 microns to about 1.0 microns, about 0.01 microns, about 0.1 microns, about 0.2 microns, about 0.3 microns, about 0.4 microns, about 0.5 microns, about 0.6 microns, about 0.7 microns, about 0.8 microns, about 0.9 microns, or about 0.95 microns.

4. The method of claim 1, wherein the ARDS is hyper-inflammatory ARDS.

5. The method of claim 1, wherein the inflammation is inflammation associated with a cytokine storm.

6. The method of claim 1, wherein the inflammation is interstitial inflammation.

7. The method of claim 1, wherein the inflammation is alveolar inflammation.

8. The method of claim 1, wherein the method comprises inhibiting at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10%, at least about 5%, of the inflammation.

9. The method of claim 1, wherein the inflammation is treated, minimized and/or inhibited by an amount at least substantially equivalent to an amount that inflammation is treated, minimized and/or inhibited following administration by inhalation of a composition containing the same amount of a corticosteroid selected from among the group consisting of prednisone, prednisolone acetate, methylprednisolone, hydrocortisone, fludrocortisone, and dexamethasone.

10. The method of claim 1, wherein administration of the composition results in reduced severity of a side effect compared to the severity of the side effect following administration by inhalation of a composition containing the same amount of a corticosteroid selected from among the group consisting of prednisone, prednisolone acetate, methylprednisolone, hydrocortisone, fludrocortisone, and dexamethasone.

11. The method of claim 10, wherein the side effect is selected from the group consisting of hyperglycemia, secondary infection, psychiatric effects, avascular necrosis, and adrenal suppression.

12. The method of claim 1, wherein said subject is infected with a COVID-19 virus.

13. The method of claim 12, wherein the COVID-19 virus is SARS-CoV-2.

14. The method of claim 12, wherein the subject is one who has been diagnosed with COVID-19.

15. The method of claim 14, wherein the composition is administered within about 24 hours, about 12 hours, about 6 hours, about 3 hours, about 1 hour, about 30 minutes, about 15 minutes, or about 5 minutes after the diagnosis of COVID-19.

16. The method of claim 1, wherein said subject suffers from non-COVID-19 ARDS.

17. The method of claim 16, wherein the non-COVID-19 ARDS is associated with a condition selected from the group consisting of sepsis; inhalation of a harmful substance; severe pneumonia; viral infection, head, chest, or other major injury; pancreatitis; fat embolism; and lung transplantation.

18. The method of claim 1, wherein the composition is administered prior to onset of severe lung symptoms.

19. The method of claim 1, wherein the subject is one whose oxygenation of blood is at or above 90%.

20. The method of claim 1, wherein the subject is one whose oxygenation of blood is below 90%.

21. The method of claim 1, wherein the LE is co-administered with an antibiotic.

22. The method of claim 21, wherein the antibiotic is selected from the group consisting of quinolone antibiotics, aminoglycosides, beta-lactams/beta-lactamase inhibitors, tetracyclines, fluoroquinolones, cephalosporins, carbapenems, and macrolide antibiotics.

23. The method of claim 21, wherein the antibiotic is selected from the group consisting of ciprofloxacin, ofloxacin, gemifloxacin, delafloxacin, tobramycin, carbenicillin, penicillin G, ticarcillin, ampicillin, nafcillin, cloxacillin, mezlocillin, oxacillin, piperacillin, azithromycin, amoxicillin, gentamicin and tobramycin, tetracycline, doxycycline, lymecycline, moxifloxacin, ceftriaxone, ornidazole, meropenem, piperacillin, tazobactam, piperacillin and tazobactam, vancomycin, ceftaroline, fluoroquinolone, and clarithromycin.

24. The method of claim 21, wherein the antibiotic is administered by a route selected from the group consisting of systemic, oral and intravenous.

25. The method of claim 21, wherein the antibiotic is administered before, after, or simultaneously with the composition comprising the LE.

26. The method of claim 1, wherein the LE is co-administered with a corticosteroid.

27. The method of claim 26, wherein the corticosteroid is selected from the group consisting of prednisone, prednisolone acetate, methylprednisolone, hydrocortisone, fludrocortisone, dexamethasone, budesonide, ciclesonide, clobetasol propionate, fluticasone propionate, flunisolide, and betamethasone dipropionate.

28. The method of claim 26, wherein the corticosteroid is administered by a route selected from the group consisting of systemic, oral, and intravenous.

29. The method of claim 26, wherein the corticosteroid is administered before, after, or simultaneously with the composition comprising the LE.

30. The method of claim 1, wherein the LE is co-administered with an enhancer selected from the group consisting of cortienic acid, delta-1 cortienic acid, delta-1 cortienic acid methyl ester, and delta-1 cortienic acid ethyl ester.

31. The method of claim 1, wherein LE is co-administered with an enhancer selected from the group consisting of hydrocortisone and hydrocortisone acetate.

32. The method of claim 30, wherein the enhancer is a micronized enhancer or a submicronized enhancer.

33. The method of claim 30, wherein the enhancer is present in the composition comprising the LE.

34. The method of claim 30, wherein the enhancer is formulated in a separate composition.

35. The method of claim 30, wherein the enhancer is administered before, after, or simultaneously with the composition comprising the LE.

36. The method of claim 1, wherein the LE, and the co-administered enhancer, when it is present, are delivered to the lungs via a metered dose inhaler (MDI).

37. The method of claim 1, wherein the LE, and the co-administered enhancer when it is present, are delivered to the lungs via a dry powder inhaler (DPI).

38. The method of claim 1, wherein the LE, and the co-administered enhancer, when it is present, are delivered to the lungs via a nebulizer.

39. The method of claim 38, wherein the composition does not comprise a preservative.

40. A pharmaceutical composition, comprising: (a) loteprednol etabonate (LE) in an amount effective to treat, prevent, minimize and/or substantially inhibit inflammation associated with COVID-19 and/or acute respiratory distress syndrome (ARDS) in a mammalian subject;
and (b) a non-toxic pharmaceutically acceptable carrier therefor suitable for lung delivery by inhalation, wherein the pharmaceutical composition is formulated for administration to the lungs of the mammalian subject by inhalation.

41. The pharmaceutical composition of claim 40, wherein the LE is micronized LE or submicronized LE.

42. The pharmaceutical composition of claim 40, wherein the average particle size of the LE is about 0.01 to about 0.1 microns, about 0.1 microns to about 0.2 microns, about 0.2 microns to about 0.3 microns, about 0.3 microns to about 0.4 microns, about 0.4 microns to about 0.5 microns, about 0.5 microns to about 0.6 microns, about 0.6 microns to about 0.7 microns, about 0.7 microns to about 0.8 microns, about 0.8 microns to about 0.9 microns, about 0.9 microns to about 0.95 microns, about 0.95 microns to about 1.0 microns, about 0.01 microns, about 0.1 microns, about 0.2 microns, about 0.3 microns, about 0.4 microns, about 0.5 microns, about 0.6 microns, about 0.7 microns, about 0.8 microns, about 0.9 microns, or about 0.95 microns.

43. The pharmaceutical composition of claim 40, wherein the ARDS is hyper-inflammatory ARDS.

44. The pharmaceutical composition of claim 40, wherein the inflammation is inflammation associated with a cytokine storm.

45. The pharmaceutical composition of claim 40, wherein the inflammation is interstitial inflammation.

46. The pharmaceutical composition of claim 40, wherein the inflammation is alveolar inflammation.

47. The pharmaceutical composition of claim 40, wherein the LE is present in an amount effective to inhibit at least about 95%, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20%, at least about 15%, at least about 10%, at least about 5%, of the inflammation.

48. The pharmaceutical composition of claim 40, wherein the LE is present in an amount effective to treat, minimize and/or inhibit inflammation by an amount at least substantially equivalent to an amount that inflammation is treated, minimized and/or inhibited following administration by inhalation of a composition containing the same amount of a corticosteroid selected from among the group consisting of prednisone, prednisolone acetate, methylprednisolone, hydrocortisone, fludrocortisone, and dexamethasone.

49. The pharmaceutical composition of claim 40, wherein the LE is present in an amount effective to treat, minimize and/or inhibit severity of a side effect compared to the severity of the side effect following administration by inhalation of a composition containing the same amount of a corticosteroid selected from among the group consisting of prednisone, prednisolone acetate, methylprednisolone, hydrocortisone, fludrocortisone, and dexamethasone.

50. The pharmaceutical composition of claim 49, wherein the side effect is selected from the group consisting of hyperglycemia, secondary infection, psychiatric effects, avascular necrosis, and adrenal suppression.

51. The pharmaceutical composition of claim 40, further comprising an enhancer selected from the group consisting of cortienic acid, delta-1 cortienic acid, delta-1 cortienic acid methyl ester, and delta-1 cortienic acid ethyl ester.

52. The pharmaceutical composition of claim 40, further comprising an enhancer selected from the group consisting of hydrocortisone and hydrocortisone acetate.

53. The pharmaceutical composition of claim 51, wherein the enhancer is a micronized enhancer or submicronized enhancer.

54. The pharmaceutical composition of claim 40, formulated as a metered dose inhaler (MDI).

55. The pharmaceutical composition of claim 40, formulated as a dry powder inhaler (DPI).

56. The pharmaceutical composition of claim 40, formulated as a nebulizer.

57. The pharmaceutical composition of claim 56, wherein the pharmaceutical composition does not comprise a preservative.

58. A combination, comprising the pharmaceutical composition of claim 40, and an antibiotic.

59. A combination, comprising the pharmaceutical composition of claim 40, and a corticosteroid.

60. A combination, comprising the pharmaceutical composition of claim 40, an antibiotic, and a corticosteroid.

61. The combination of claim 58, wherein the antibiotic is selected from the group consisting of quinolone antibiotics, aminoglycosides, beta-lactams/beta-lactamase inhibitors, tetracyclines, fluoroquinolones, cephalosporins, carbapenems, and macrolide antibiotics.

62. The combination of claim 58, wherein the antibiotic is selected from the group consisting of ciprofloxacin, ofloxacin, gemifloxacin, delafloxacin, tobramycin, carbenicillin, penicillin G, ticarcillin, ampicillin, nafcillin, cloxacillin, mezlocillin, oxacillin, piperacillin, azithromycin, amoxicillin, gentamicin and tobramycin, tetracycline, doxycycline, lymecycline, moxifloxacin, ceftriaxone, ornidazole, meropenem, piperacillin, tazobactam, piperacillin and tazobactam, vancomycin, ceftaroline, fluoroquinolone, and clarithromycin.

63. The combination of claim 58, wherein the antibiotic is formulated for administration by a route selected from the group consisting of systemic, oral and intravenous.

64. The combination of claim 58, wherein the antibiotic is formulated for administration before, after, or simultaneously with the composition comprising the LE.

65. The combination of claim 59, wherein the corticosteroid is selected from the group consisting of prednisone, prednisolone acetate, methylprednisolone, hydrocortisone, fludrocortisone, dexamethasone, budesonide, ciclesonide, clobetasol propionate, fluticasone propionate, flunisolide, and betamethasone dipropionate.

66. The combination of claim 59, wherein the corticosteroid is formulated for administration by a route selected from the group consisting of systemic, oral, and intravenous.

67. The combination of claim 59, wherein the corticosteroid is formulated for administration before, after, or simultaneously with the composition comprising the LE.

68. A method of treating, preventing, minimizing and/or substantially inhibiting inflammation associated with COVID-19 and/or acute respiratory distress syndrome (ARDS) in a mammalian subject in need of such treatment, said method comprising administering to the lungs of said subject, by inhalation, a composition comprising: (a) an effective amount of a therapeutic agent; and (b) a non-toxic pharmaceutically acceptable carrier therefor suitable for lung delivery by inhalation, wherein the therapeutic agent is selected from the group consisting of a compound of formula (I), a compound of formula (III), loteprednol etabonate (LE), and a combination thereof 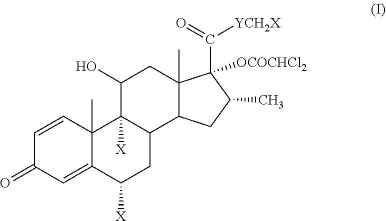
wherein, in Formula (I), each X is independently F or Cl, and Y is O or S; and 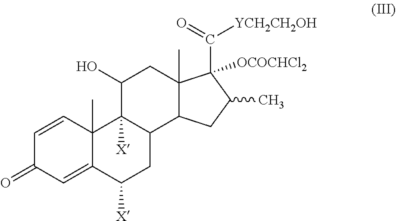
wherein, in Formula (III), X′ is independently H, F or Cl provided that at least one X′ is F or Cl, Y is O or S, and the wavy line indicates the α- or β-configuration.
